(19) United States
(12) Patent Application Publication
(10) Pub. No.: US 2012/0041070 A1
(43) Pub. Date: Feb. 16, 2012

(54) METHODS OF TREATING CANCER HAVING AN ABERRANT EGFR OR KRAS GENOTYPE

(76) Inventors: Shengfang Jin, Newton, MA (US); Shinsan Su, Newton, MA (US); Matthew Van Der Heiden, Belmont, MA (US)

(21) Appl. No.: 13/249,609

(22) Filed: Sep. 30, 2011

Related U.S. Application Data
(63) Continuation of application No. PCT/US2010/029470, filed on Mar. 31, 2010.
(60) Provisional application No. 61/165,387, filed on Mar. 31, 2009.

(51) Int. Cl. A61K 31/155; A61P 35/00; C12N 5/02

(52) U.S. Cl. 514/635; 435/375

(57) ABSTRACT

The invention relates to the treating cancer having an aberrant EGFR or KRAS genotype, with an AMPK activator, as well as related methods, compounds and compositions.

### CLAIM OF PRIORITY

This application claims priority from U.S. Ser. No. 61/165,387, filed Mar. 31, 2009, which is incorporated herein by reference in its entirety.

### BACKGROUND

Historically anti-cancer therapies have often relied attempts to directly distinguish cancer from non-cancerous cells by their rates of cell division. There is a long felt need for additional approaches.

### SUMMARY OF THE INVENTION

In general, the invention features, a method of treating a subject having cancer having an aberrant EGFR or KRAS genotype (e.g., mutation(s), unwanted expression (e.g., overexpression) and/or amplification of EGFR or KRAS). The method includes administering, to the subject, a therapeutically effective amount of an AMPK activator, e.g., a direct activator (e.g., AICAR or a compound of formula (I) or (II) described herein) or indirect activator (e.g., a biguanide (e.g., phenformin) of AMPK. In an embodiment the cancer is a cancer disclosed herein.

In some embodiments the activator is a direct activator while in others it is an indirect activator.

In some embodiments the direct or indirect AMPK activator will be administered as part of a combination therapy, i.e., it will be administered in conjunction with another anti-cancer agent.

In another aspect, the invention features, a method of promoting apoptosis of a cell having an aberrant EGFR or KRAS genotype (e.g., mutation(s), unwanted expression (e.g., overexpression) and/or amplification of EGFR or KRAS). The method includes contacting the cell with an AMPK activator, e.g., a direct AMPK activator (e.g., AICAR or a compound of formula (I) or (II) described herein) or an indirect AMPK activator (e.g., a biguanide (e.g., phenformin)).

In one aspect, the invention features, a method of treating a patient suffering from a cancer characterized by cells having an aberrant genotype for EGFR or KRAS (e.g., mutation(s), unwanted expression (e.g., overexpression) and/or amplification of EGFR or KRAS). The method includes administering to the patient in need thereof a therapeutically effective amount of a pharmaceutical composition comprising an AMPK activator, e.g., phenformin, and a pharmaceutically acceptable carrier. The AMPK activator can be a direct or indirect activator. In an embodiment the AMPK activator is AICAR or a compound of formula (I) or (II) as described herein. In an embodiment the AMPK activator is a biguanide, e.g., phenformin.

In an embodiment the method includes the additional step of evaluating the cancer for an aberrant EGFR or KRAS genotype, e.g., prior to the administration of the activator of AMPK such as phenformin. The result of this evaluation can be used to select a treatment, e.g., to select an AMPK activator, e.g., phenformin, to use in the treatment of the subject. The evaluation can include direct DNA sequencing or immunohistochemical analysis.

In an embodiment the cancer is characterized by unwanted (e.g., unimpaired) glycolysis (e.g., prior to the administration of an activator of AMPK). Unwanted glycolysis, as used herein, refers to glycolysis which supports glycolysis dependent growth of a cancer cell. Thus, cancer cells that have glycolysis dependent growth have unwanted glycolysis.

In an embodiment the cancer is characterized by unwanted (e.g., unimpaired) glutamine uptake (e.g., prior to the administration of an activator of AMPK). Unwanted glutamine uptake, as used herein, refers to glutamine uptake which supports glutamine dependent growth of a cancer cell. Thus, cancer cells that have glutamine dependent growth have unwanted glutamine uptake.

In an embodiment the method includes subjecting the patient in need thereof to a second anti-cancer therapeutic treatment selected from radiation therapy, chemotherapy, hormonal therapy, or treatment with a targeted cancer compound.

In an embodiment the patient is suffering from at least one tumor caused by the cancer.

In an embodiment the aberrant genotype is in the EGFR or KRAS gene and the cancer is cancer of the colon, lung or pancreas.

In an embodiment the aberrant genotype is in the EGFR or KRAS gene and the cancer is cancer of the prostate, breast or brain (e.g., a glioblastoma).

In an embodiment the method includes subjecting the patient in need thereof to a second anti-cancer therapeutic treatment selected from radiation therapy, chemotherapy, hormonal therapy, or treatment with a targeted cancer compound. Exemplary cancer treatments include taxotere, mitoxantrone, anti-androgen therapy, radiation, anti-estrogen therapy, xeloda, 5-FU, adriamycin, cytoxan, taxol, taxotere, avastin, herceptin, temodar, and tarceva.

In an embodiment the cancer has an aberrant genotype for EGFR or KRAS, the cancer is selected from colon, lung or pancreas cancer and method includes second anti-cancer therapeutic treatment comprising gemcitabine, cis/carbo-platinum, pemetrexed, oxaliplatin, and irinotecan or a combination thereof.

In an embodiment the cancer has an aberrant genotype for EGFR or KRAS, the cancer is selected from prostate, breast or brain cancer (e.g., glioblastoma) and method includes second anti-cancer therapeutic treatment comprising radiation, taxotere, mitoxantrone, anti-androgen therapy, anti-estrogen therapy, xeloda, 5-FU, adriamycin, cytoxan, taxol, taxotere, avastin, herceptin, temodar, and tarceva or a combination thereof.

In another aspect, the invention features, a method of promoting apoptosis of a cell having an aberrant EGFR or KRAS genotype (e.g., mutation(s), unwanted expression (e.g., overexpression) and/or amplification of EGFR or KRAS). The method includes contacting the cell with an AMPK activator, e.g., a direct AMPK activator or an indirect AMPK activator (e.g., a biguanide (e.g., phenformin)) Other steps, such as those described in the section above on treating a patient, can be adapted and included with this method.

In another aspect, the invention features, a pharmaceutical composition for treating a cancer having an aberrant EGFR or KRAS genotype (e.g., mutation(s), unwanted expression (e.g., overexpression) and/or amplification of EGFR or KRAS). It includes: an AMPK activator, e.g., a direct AMPK activator or an indirect AMPK activator (e.g., a biguanide (e.g., phenformin)) an anti-cancer agent; and a pharmaceutically acceptable carrier.

In another aspect, the invention features, a kit. The kit includes one or more of the following:

a first vessel containing a reagent or component for assaying a cancer for aberrant EGFR or KRAS genotype (e.g., mutation(s), unwanted expression (e.g., overexpression) and/or amplification of EGFR or KRAS);

a second vessel containing a pharmaceutically acceptable composition comprising an AMPK activator, e.g., a direct AMPK activator or an indirect AMPK activator (e.g., a biguanide (e.g., phenformin)) and a pharmaceutically acceptable carrier;

instructions for using said kit to perform an assay to determine whether a cancer has an aberrant EGFR or KRAS genotype; and

instructions to administer to a subject having the cancer that has aberrant EGFR or KRAS genotype an AMPK activator, e.g., a direct AMPK activator (e.g., AICAR) or an indirect AMPK activator (e.g., a biguanide (e.g., phenformin)).

In another aspect, the invention features, a method of selecting a patient for treatment, e.g., a patient suffering from a cancer characterized by cells that has an aberrant EGFR or KRAS genotype (e.g., mutation(s), unwanted expression (e.g., overexpression) and/or amplification of EGFR or KRAS). The method includes:

selecting a patient on the basis of the patient having cancer cells that have aberrant EGFR or KRAS genotype;

optionally, selecting a drug, e.g., phenformin, e.g., on the basis that the drug is useful for treating cancer that has an aberrant EGFR or KRAS genotype; and

optionally, administering to the patient in need thereof a therapeutically effective amount of a pharmaceutical composition comprising an AMPK activator, e.g., a direct AMPK activator or an indirect AMPK activator (e.g., a biguanide (e.g., phenformin)) and a pharmaceutically acceptable carrier.

A method of selecting a drug for treating a patient suffering from a cancer characterized by cells that have an aberrant EGFR or KRAS status (e.g., mutation(s), unwanted expression (e.g., overexpression) and/or amplification of EGFR or KRAS), comprising:

optionally, selecting an activator of AMPK e.g., phenformin, e.g., on the basis that the activator of AMPK is useful for treating cancer that has an aberrant EGFR or KRAS status (e.g., mutation(s), unwanted expression (e.g., overexpression); and

optionally, administering to the patient in need thereof a pharmaceutical composition comprising an AMPK activator, e.g., a direct AMPK or an indirect AMPK activator (e.g., a biguanide (e.g., phenformin or metformin)) and a pharmaceutically acceptable carrier.

A method of selecting a patient for treatment, e.g., a patient suffering from a cancer, and selecting a drug for treating said patient comprising:

selecting a patient on the basis of the patient having cancer cells that have an aberrant EGFR or KRAS status (e.g., mutation(s), unwanted expression (e.g., overexpression) and/or amplification of EGFR or KRAS);

selecting a drug, e.g., an AMPK activator, e.g., a direct AMPK activator or an indirect AMPK activator (e.g., a biguanide (e.g., phenformin or metformin)), e.g., on the basis that the drug is useful for treating cancer that has an aberrant EGFR or KRAS status (e.g., mutation(s), unwanted expression (e.g., overexpression) and/or amplification of EGFR or KRAS); and

optionally, administering to the patient in need thereof a pharmaceutical composition comprising an AMPK activator, e.g., a direct AMPK activator or an indirect AMPK activator (e.g., a biguanide (e.g., phenformin or metformin)) and a pharmaceutically acceptable carrier.

In one aspect, the invention features, a method of treating a patient suffering from a cancer characterized by cells having an aberrant genotype for EGFR or KRAS (e.g., mutation(s), unwanted expression (e.g., overexpression) and/or amplification of EGFR or KRAS). The method includes administering to the patient in need thereof a therapeutically effective amount of a pharmaceutical composition comprising phenformin, and a pharmaceutically acceptable carrier. In an embodiment the cancer has an aberrant genotype for EGFR or KRAS, the cancer is selected from colon, lung or pancreas cancer and method includes second anti-cancer therapeutic treatment comprising gemcitabine, cis/carbo-platinum, pemetrexed, oxaliplatin, and irinotecan or a combination thereof. In an embodiment the cancer has an aberrant genotype for EGFR or KRAS, the cancer is selected from prostate, breast or brain cancer (e.g., glioblastoma) and method includes second anti-cancer therapeutic treatment comprising radiation, taxotere, mitoxantrone, anti-androgen therapy, anti-estrogen therapy, xeloda, 5-FU, adriamycin, cytoxan, taxol, taxotere, avastin, herceptin, temodar, and tarceva or a combination thereof.

### BRIEF DESCRIPTION OF THE DRAWINGS

FIG. 1 depicts the inhibitory effect of phenformin on the proliferation of KRAS mutated cancer cell line A549 NSCL.

### DESCRIPTION

As used herein, the term “cancer”, is meant to include all types of cancerous growths or oncogenic processes, metastatic tissues or malignantly transformed cells, tissues, or organs, irrespective of histopathologic type or stage of invasiveness. Treatment of cancer includes, e.g., slowing, eliminating, or reversing tumor growth, preventing or reducing, either in number or size, metastases, reducing or eliminating tumor cell invasiveness, providing an increased interval to tumor progression, or increasing disease-free survival time.

An “effective amount” refers to an amount which, when administered in a proper dosing regimen, is sufficient to reduce or ameliorate the severity, duration or progression of the disorder being treated (e.g., cancer), prevent the advancement of the disorder being treated (e.g., cancer), cause the regression of the disorder being treated (e.g., cancer), or enhance or improve the prophylactic or therapeutic effects(s) of another therapy (e.g., cancer). An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents.

### AMPK

AMPK is adenosine monophosphate-activated protein kinase, which consists of three protein subunits that together make a functional enzyme, conserved from yeast to humans, that plays a role in cellular energy homeostasis. It is expressed in a number of tissues. The net effect of AMPK activation is generally stimulation of hepatic fatty acid oxidation and ketogenesis, inhibition of hepatic cholesterol synthesis, lipogenesis, and triglyceride synthesis, inhibition of adipocyte lipolysis and lipogenesis, stimulation of skeletal muscle fatty acid oxidation and muscle glucose uptake, and modulation of insulin secretion by pancreatic beta-cells.

### AMPK Activators

Activators of AMPK result in a direct or indirect increase in activity of AMPK. Direct activators of AMPK interact, e.g., bind, directly with AMPK to activate. Indirect activators can interact, for example, with a member of the “mitochondrial complex 1” to activate AMPK.

Examples of direct activators include AICAR and the compounds of formula (I) and (II) described herein

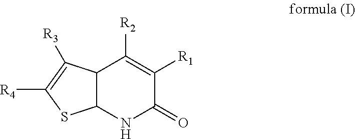

or a therapeutically acceptable salt or prodrug thereof, wherein

R₁ is selected from the group consisting of hydrogen, alkoxy, alkoxycarbonyl, alkyl, alkenyl, alkynyl, carboxy, carboxyalkyl, cyano, cyanoalkyl, halogen, haloalkyl, trihaloalkyl, heterocycle, hydroxyalkyl, RₐR_{b}N—, RₐR_{b}Nalkyl, and R_{c}R_{d}NC(O)—, wherein alkyl may be optionally substituted with O— and Rₜ—N—;

R₂ is selected from the group consisting of R_{f}O—, HO—, R_{f}S—, and HS—;

R₃ is selected from the group consisting of alkoxycarbonyl, substituted aryl, arylalkyl, arylalkenyl, arylalkynyl, cycloalkyl, carboxy, carboxylalkyl, halogen, heteroaryl, heterocycle, heterocyclealkyl, R_{g}RⱼN—, and R_{g}RⱼNalkyl, wherein cycloalkyl may be fused to an aryl ring as defined herein;

R₄ is selected from the group consisting of hydrogen, alkyl, alkenyl, alkynyl, aryl, arylalkyl, arylcarbonyl, aryloxycarbonyl, carboxy, carboxyalkyl, carboxyalkynyl, halogen, haloalkyl, heteroaryl, heterocycle, heterocyclealkyl, heterocyclecarbonyl, heterocycleoxycarbonyl, hydroxyalkyl, HO—N—CH—(CH₂)ᵤ—, and RₘRₙN—;

u is 0, 1 or 2;

Rₐ and R_{b} are each individually selected from the group consisting of hydrogen, alkyl, alkylcarbonyl, alkoxylcarbonyl, aryl, arylalkyl, arylcarbonyl, arylalkyloxycarbonyl, heteroaryl, heterocycle, heterocyclealkyl, heterocyclecarbonyl, and heterocycleoxycarbonyl;

R_{c} and R_{d} are each independently selected from the group consisting of hydrogen, alkyl, aryl, arylalkyl, haloalkyl, hydroxyalkyl, and heterocycle;

R_{f} is selected from the group consisting of alkyl, cycloalkyl, cycloalkylalkyl, alkoxyalkyl, alkylthioalkyl, and haloalkyl;

R_{g} and Rⱼ are each independently selected from the group consisting of hydrogen, alkyl, alkoxycarbonyl, aryl, arylalkyl, arylcarbonyl, aryloxycarbonyl, heteroaryl, heterocycle, heterocyclealkyl, heterocyclecarbonyl, heterocycleoxycarbonyl, and haloalkyl;

Rₘ and Rₙ are each independently selected from the group consisting of hydrogen, alkyl, alkoxycarbonyl, aryl, arylalkyl, heteroaryl, heterocycle, heterocyclealkyl, and haloalkyl; and

Rₜ is selected from the group consisting of hydrogen, alkyl and HO—.

An exemplary compound of formula (I) is the following compound of formula (II)

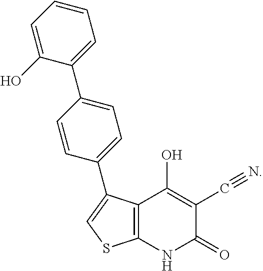

Additional candidate direct activators can be found in US 2005/0038068, which is incorporated herein by reference.

Examples of indirect activators include the biguanide class of molecules. Biguanide is shown below

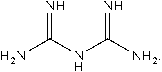

Exemplary biguanides include compounds wherein one or more of the hydrogen moieties of the biguanide have been replaced with a functional group such as an alkyl or arylalkyl moiety e.g., metformin, buformin or phenformin.

### Phenformin

The AMPK activator phenformin is of particular interest. Phenformin is a biguanide compound having the structure provided below:

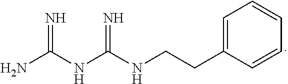

Phenformin has been used to treat diabetes, for example, type 2 diabetes. Phenformin can be used in method of the invention to treat any of the cancers described herein. It can be used to treat subjects having an aberrant EGFR or KRAS genotype. It can be administered in combination with other anti-cancer treatments. Methods and dosages of phenformin in the treatment of cancer can be evaluated in an animal model, for example, a mouse model, as described, e.g., in Arteaga C. et al., Cancer Cell, Volume 9, Issue 6, Pages 421-423.

Additional candidate AMPK activators can be evaluated for suitability for use in methods described in US 2005/0038068, e.g., by using a protocol or modified version thereof described by Davies et. al. (Zhou, M. et.al. UCP-3 expression in skeletal muscle: effects of exercise, hypoxia, and AMP-activated protein kinase. Am. J. Physiol. Endocrinol. Metab. 279: E622 (2000)).

### EGFR Aberrant Cancers

As used herein, “EGFR” refers to epidermal growth factor receptor (erythroblastic leukemia viral (v-erb-b) oncogene homolog, avian). EGFR is also known in the art as ERBB, HER1, mENA, ERBB1 and PIG61. The protein encoded by this gene is a transmembrane glycoprotein that is a member of the protein kinase superfamily. This protein is a receptor for members of the epidermal growth factor family. EGFR is a cell surface protein that binds to epidermal growth factor. Binding of the protein to a ligand induces receptor dimerization and tyrosine autophosphorylation and leads to cell proliferation. Mutations in this gene are associated with diseases, e.g., lung cancer (non-small cell lung cancer (NSCLC)), head and neck cancer, glioblastoma and pancreatic cancer.

As used herein, aberrant EGFR means that sufficient cells of the tumor have one or more mutations in EGFR (e.g., an activating mutation), unwanted expression of EGFR (e.g., overexpression over wild type), EGFR deficiency, and/or amplification of EGFR gene (e.g., having more than two functional copies of EGFR gene). In embodiments sufficient cells of the cancer have one or more mutations in EGFR, such that a sample of cancer cells can be distinguished from a sample of non-cancerous cells of the same tissue, e.g., by a growth or morphological phenotype, or by the number of cells having one or more mutations in EGFR. In embodiments at least 2, 5, 10, 20, 30, 40, or 50% of the cells in a tumor have aberrant EGFR status. In some embodiments, at least 2, 5, 10, 20, 30, 40, or 50 of the cells in a microliter of blood have aberrant EGFR status.

In some embodiments, the aberrant status of EGFR results in unwanted expression (e.g., overexpression), and/or amplification of EGFR.

Mutations in this gene are associated with diseases, e.g., lung cancer (non-small cell lung cancer (NSCLC)), head and neck cancer, glioblastoma and pancreatic cancer.

As discussed herein, AMPK activators can be used to treat subjects having a cancer characterized by a mutation in EGFR. Such cancers include a cancer described herein (e.g., a cancer having aberrant EGFR genotype).

A subject having one of these cancers can be treated by administering a combination of an AMPK activator (direct or indirect) and another treatment such as Tarceva, Cisplatin (or Carboplatin), Taxol, Taxotere, Gemcitabine, Navelbine, Alimta (pemetrexed). Exemplary combinations include phenformin and Cisplatin (or Carboplatin), 5-FU, Xeloda, cetuximab, and/or radiation in the treatment of head and neck cancer; phenformin and Temodar, Tarceva, and/or Radiation in the treatment of glioblastoma; and phenformin and gemcitabine, 5-FU, Xeloda, and/or Tarceva in the treatment of pancreatic cancer.

### KRAS Aberrant Cancers

As used herein, “KRAS” refers to v-Ki-ras2 Kirsten rat sarcoma viral oncogene homolog. KRAS is also known in the art as NS3, KRAS1, KRAS2, RASK2, KI-RAS, C-K-RAS, K-RAS2A, K-RAS2B, K-RAS4A and K-RAS4B. This gene, a Kirsten ras oncogene homolog from the mammalian ras gene family, encodes a protein that is a member of the small GTPase superfamily. A single amino acid substitution can be responsible for an activating mutation. The transforming protein that results can be implicated in various malignancies, including lung cancer, colon cancer and pancreas cancer.

As used herein, aberrant KRAS means that sufficient cells of the tumor have one or more mutations in KRAS (e.g., an activating mutation), unwanted expression of KRAS (e.g., overexpression over wild type), KRAS deficiency, and/or amplification of KRAS gene (e.g., having more than two functional copies of KRAS gene). In some embodiments at least 2, 5, 10, 20, 30, 40, or 50% of the cells in a tumor have aberrant KRAS status. In some embodiments, at least 2, 5, 10, 20, 30, 40, or 50 of the cells in a microliter of blood have aberrant KRAS status.

In some embodiments, the aberrant status of KRAS results in unwanted expression (e.g., overexpression), and/or amplification of KRAS.

Mutations in this gene are associated with diseases, e.g., lung cancer, colon cancer and pancreas cancer.

As discussed herein, AMPK activators can be used to treat subjects having a cancer characterized by a mutation in KRAS. Such cancers include a cancer described herein (e.g., a cancer having aberrant KRAS genotype).

A subject having one of these cancers can be treated by administering a combination of an AMPK activator (direct or indirect) and another treatment such as Cisplatin (or Carboplatin), Taxol, Taxotere, Gemcitabine, Navelbine, Alimta, (pemetrexed), Avastin, Tarceva, Gemcitabine, 5-FU, Xeloda, irinotecan, oxaliplatin, and/or cetuximab. Exemplary combinations include phenformin and Cisplatin (or Carboplatin), Taxol, Taxotere, Gemcitabine, Navelbine, Alimta, (pemetrexed), Avastin, and/or Tarceva in the treatment of Lung cancer; phenformin and Gemcitabine, 5-FU, Xeloda, and/or Tarceva in the treatment of pancreatic cancer; and phenformin and 5-FU, Xeloda, irinotecan, oxaliplatin, cetuximab, and/or avastin in colon cancer.

### Disorders

AMPK activators (direct or indirect) can be used to treat subjects having a cancer having an aberrant EGFR or KRAS genotype, e.g., a cancer disclosed herein. In some embodiments, the AMPK activator is phenformin. A cancer can be assayed for the presence of an aberrant EGFR or KRAS genotype using methods known in the art or described herein.

The disclosed methods are useful in the prevention and treatment of solid tumors, soft tissue tumors, and metastases thereof wherein the solid tumor, soft tissue tumor or metastases thereof is a cancer described herein (e.g., a cancer having an aberrant EGFR or KRAS).

The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

The methods described herein can be administered with any cancer, for example those described by the national cancer institute. A cancer can be evaluated to determine whether it is EGFR or KRAS aberrant using methods known in the art, including methods described herein. Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

In some embodiments, the cancer is lung cancer (non-small cell lung cancer) head and neck cancer, glioblastoma, or pancreatic cancer.

### Patient Selection

The methods described herein include methods of administering an AMPK activator to a subject wherein the subject has a cancer characterized as having an aberrant genotype for EGFR or KRAS (e.g., mutation(s), unwanted expression (e.g., overexpression) and/or amplification of EGFR or KRAS).

The methods described herein include administering an AMPK activator to a subject wherein the subject has a cancer having aberrant genotype for EGFR or KRAS. A cancer can be evaluated to determine whether it has an aberrant genotype (e.g., mutation(s), unwanted expression (e.g., overexpression) and/or amplification of EGFR or KRAS) using methods known in the art. Accordingly, described herein are methods of selecting a subject (or patient) to be treated with an AMPK activator. The method includes evaluating a subject (or patient) to determine if the subject (or patient) is suffering from a cancer having an aberrant genotype for EGFR or KRAS and if the subject (or patient) is suffering from a cancer having an aberrant genotype then treating or instructing to treat the subject with an AMPK activator.

Methods are described herein for selecting a patient on the basis that the patient has a cancer having an aberrant genotype for EGFR or KRAS, and administering an AMPK activator to that patient. Methods are also described for selecting a pharmaceutical agent (e.g., a drug) for treating a subject suffering from cancer, for example, a cancer having an aberrant genotype for EGFR or KRAS. The method includes evaluating a subject suffering from cancer to determine whether the cancer has an aberrant genotype for EGFR or KRAS, and if the cancer has the aberrant genotype, selecting an AMPK activator to treat the subject (e.g., selecting phenformin on the basis that the cancer has an aberrant genotype for EGFR or KRAS). Exemplary methods of determining whether a cancer has an aberrant genotype are provided herein.

Exemplary methods for evaluating a cancer to determine whether the cancer has an aberrant genotype include the following: evaluating a subject for an aberrant EGFR or KRAS genotype, for example, by direct DNA sequencing or immunohistochemistry (IHC). Other exemplary methods for evaluating a subject for having an aberrant EGFR or KRAS genotype include the following: Single-stranded conformation polymorphism (SSCP), for example, as described in Lee J W et al., Clin Cancer Res. 2005; 11(8):2879-82; Denaturing gradient gel electrophoresis (DGGE), for example, as described in Vermeij J et al., BMC Cancer 2008; 8:3; Denaturing high-performance liquid chromatography (DHPLC), for example, as described in Cohen V et al., Cancer. 2006; 107(12):2858-65; Heteroduplex detection, for example, as described in Oshita F et al., Br J Cancer. 2006; 95(8):1070-5; Functional analysis of separated alleles in yeast (FASAY), for example, as described in Rand A et al., Cancer Lett. 1996; 98(2):183-91; Oligonucleotide array/DNA chip, for example, as described in Tsukamoto Y, et al., J Pathol. 2008; 216(4):471-82; Fluorescence in situ hybridization (FISH), for example, as described in Gevorgyan A et al., J. Clin Oncology 2007, 25(18S): 21070; and Biosensors e.g., Surface plasmon resonance (SPR), for example, as described in Haga Y et al., Carbohydr Res. 2008; 343(18):3034-8.

### Combination Therapies

In some embodiments, a direct or indirect AMPK activator, e.g., phenformin, is administered together with an additional cancer treatment. Exemplary cancer treatments include, for example: chemotherapy, targeted therapies such as antibody therapies, immunotherapy, and hormonal therapy. Examples of each of these treatments are provided below.

Chemotherapy

In some embodiments, direct or indirect AMPK activator described herein, e.g., phenformin, is administered in conjunction with a chemotherapy. Chemotherapy is the treatment of cancer with drugs that can destroy or inhibit cancer cells. “Chemotherapy” usually refers to cytotoxic drugs which affect rapidly dividing cells. Chemotherapy drugs interfere with cell division in various ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example: antimetabolites (e.g., folic acid, purine, and pyrimidine derivatives); and alkylating agents (e.g., nitrogen mustards, nitrosoureas, platinum, alkyl sulfonates, hydrazines, triazenes, aziridines, spindle poison, cytotoxic agents, topoisomerase inhibitors and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy. In some embodiments, the chemotherapy agents (including combination chemotherapy) can be used in combination with a compound described herein, e.g., phenformin

Targeted Therapy

In some embodiments, a direct or indirect AMPK activator, e.g., phenformin, is administered with a targeted therapy. Targeted therapy constitutes the use of agents specific for the cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. Monoclonal antibody therapy is another strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a compound described herein, e.g., a biguanide such as metformin or phenformin, preferably phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR® Immunotherapy.

Immunotherapy

In some embodiments, direct or indirect AMPK activator, e.g., phenformin, is administered with an immunotherapy. Cancer immunotherapy refers to a diverse set of therapeutic strategies designed to induce the patient's own immune system to fight the tumor. Contemporary methods for generating an immune response against tumors include intravesicular BCG immunotherapy for surficial bladder cancer, and use of interferons and other cytokines to induce an immune response in renal cell carcinoma and melanoma patients.

Allogeneic hematopoietic stem cell transplantation can be considered a form of immunotherapy, since the donor's immune cells will often attack the tumor in a graft-versus-tumor effect. In some embodiments, the immunotherapy agents can be used in combination with a compound described herein, e.g., phenformin.

Hormonal Therapy

In some embodiments, direct or indirect AMPK activator, e.g., phenformin, is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with direct or indirect AMPK activator, e.g., phenformin

A subject having one of these cancers can be treated by administering a combination of an AMPK activator (direct or indirect) and another treatment. Tarceva, Cisplatin (or Carboplatin), Taxol, Taxotere, Gemcitabine, Navelbine, Alimta (pemetrexed). Exemplary combinations include phenformin and Cisplatin (or Carboplatin), 5-FU, Xeloda, cetuximab, and/or radiation in the treatment of head and neck cancer; phenformin and Temodar, Tarceva, and/or Radiation in the treatment of glioblastoma; and phenformin and gemcitabine, 5-FU, Xeloda, and/or Tarceva in the treatment of pancreatic cancer.

### Compositions and Routes of Administration

The compositions delineated herein include the compounds delineated herein (e.g., phenformin), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Kits

A compound described herein described herein can be provided in a kit. The kit includes (a) a compound described herein, e.g., a composition that includes a compound described herein, and, optionally (b) informational material. The informational material can be descriptive, instructional, marketing or other material that relates to the methods described herein and/or the use of a compound described herein for the methods described herein. In some embodiments, the kit can include a reagent such as a probe or an antibody useful for determining if a subject has an aberrant EGFR or KRAS genotype.

In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

### Example 1

### Phenformin is Effective at Inhibiting Cell Proliferation of a TERT-Immortalized Human Mammary Epithelial Cell Line (HME-TERT) Carrying an EGFR Mutation (del E746-A750)

To investigate the effect of phenformin on proliferation of cells carrying an EGFR mutation, HME cell proliferation assays were performed. An isogenic HME-TERT cell line carrying a mutation in EGFR (del E746-A750) was generated by a “knock in” approach. HME-TERT-EGFR (del E746-A750) cells and control HME parental cells were cultured in DMEM/F12K (glucose 17 mM, glutamine 2.5 mM) with 0.5% FBS, and were treated with a series of dilution of Paclitaxel (100 nM top concentration, 3-fold dilution), Phenformin (3 mM top concentration, 3-fold dilution), or metformin (10 mM top concentration, 3-fold dilution), in duplication or triplication. MTS assays were performed 48, 72, 96 or 120 hours after drug treatment. MTS assay is a colorimetric assay for determining the number of viable cells (see, e.g., Cory A H et al., 1991, Cancer communications 3 (7): 207-12, and Wilson, A P, Cytotoxicity and Viability Assays in Animal Cell Culture: A Practical Approach, 3rd ed. (ed. Masters, J. R. W.) Oxford University Press, 2000, Vol. 1). EC50, as shown in Table 1, was defined using XcellFit software. Z′ values, as shown in Table 2, were determined for assay quality control for each plate of cells. Z′ is defined as Z′=1-3(MAX stdev−background stdev)/(MAX−background). A Z′ value greater than 0.4 is acceptable for cell assays, and a Z′ value greater than 0.6 is acceptable for enzyme assays.

TABLE 1
Inhibition of cell proliferation: EC₅₀

| EC₅₀ (μM) | WT | EGFR | Time |
|---|---|---|---|
| phenformin | 2088 | 1677 | 48 h |
| | 1692 | 632 | 72 h |
| | 1135 | 486 | 96 h |
| | 626 | 393 | 120 h |
| metformin | >3000 | >3000 | 48 h |
| | >3000 | >3000 | 72 h |
| | >3000 | >3000 | 96 h |
| | >3000 | >3000 | 120 h |

TABLE 2
Cell assay quality control

| Z′ | WT | EGFR |
|---|---|---|
| 48 hr | 0.86 | 0.53 |
| 72 hr | 0.74 | 0.62 |
| 96 hr | 0.88 | 0.53 |
| 120 hr | 0.80 | 0.68 |

The MTS assay results showed that phenformin inhibited proliferation of TERT-immortalized HME cells carrying an activating EGFR mutation more than that of wild type cells. Phenformin is more potent than metformin in inhibition of cell proliferation in both wild type and EGFR mutated HME-TERT cells.

### Example 2

### Phenformin is Effective at Inhibiting Cell Proliferation of a TERT-Immortalized Human Mammary Epithelial Cell Line (HME-TERT) Carrying a KRAS Mutation (G13D)

To investigate the effect of phenformin on proliferation of cells carrying a KRAS mutation, HME cell proliferation assays were performed. An isogenic HME-TERT cell line carrying a mutation in KRAS (G13D) was generated by a “knock in” approach. HME-TERT-KRAS (G13D) cells and control HME parental cells were cultured in DMEM/F12K (glucose 17 mM, glutamine 2.5 mM) with 0.5% FBS, and were treated with a series of dilution of Paclitaxel (100 nM top concentration, 3-fold dilution), Phenformin (3 mM top concentration, 3-fold dilution), or metformin (10 mM top concentration, 3-fold dilution), in duplication or triplication. MTS assays were performed 48, 72, 96 or 120 hours after drug treatment. MTS assay is a colorimetric assay for determining the number of viable cells (see, e.g., Cory A H et al., 1991, Cancer communications 3 (7): 207-12, and Wilson, A P, Cytotoxicity and Viability Assays in Animal Cell Culture: A Practical Approach, 3rd ed. (ed. Masters, J. R. W.) Oxford University Press, 2000, Vol. 1). EC50, as shown in Table 3, was defined using XcellFit software. Z′ values, as shown in Table 2, were determined for assay quality control for each plate of cells. Z′ is defined as Z′=1-3(MAX stdev−background stdev)/(MAX−background). A Z′ value greater than 0.4 is acceptable for cell assays, and a Z′ value greater than 0.6 is acceptable for enzyme assays.

TABLE 3
Inhibition of cell proliferation: EC50

| EC50 (μM) | WT | KRAS | Time |
|---|---|---|---|
| phenformin | 2088 | 1640 | 48 h |
| | 1692 | 1247 | 72 h |
| | 1135 | 727 | 96 h |
| | 626 | 440 | 120 h |
| metformin | >3000 | >3000 | 48 h |
| | >3000 | >3000 | 72 h |
| | >3000 | >3000 | 96 h |
| | >3000 | >3000 | 120 h |

TABLE 4
Cell assay quality control

| Z′ | WT | KRAS |
|---|---|---|
| 48 hr | 0.86 | 0.79 |
| 72 hr | 0.74 | 0.76 |
| 96 hr | 0.88 | 0.92 |
| 120 hr | 0.80 | 0.87 |

The MTS assay results showed that phenformin inhibited proliferation of TERT-immortalized HME cells carrying an activating KRAS mutation more than that of wild type cells. Phenformin is more potent than metformin in inhibition of cell proliferation in both wild type and KRAS mutated HME-TERT cells.

### Example 3

### Phenformin is Effective at Inhibiting Cell Proliferation of Cancer Cell Line A549 NSCL

To investigate the effect of phenformin on proliferation of a commonly used cancer cell line A549 NSCL (KRAS mutant), a cell proliferation assay was performed. A549 cells were grown in DMEM with 10% FBS, and were treated with designated concentrations (phenformin dose range 0-100 μM; metformin dose range 0-3000 μM) of drugs for 72 hours. Cell viability was determined with Cell TiterGlow to measure total ATP content in live cells.

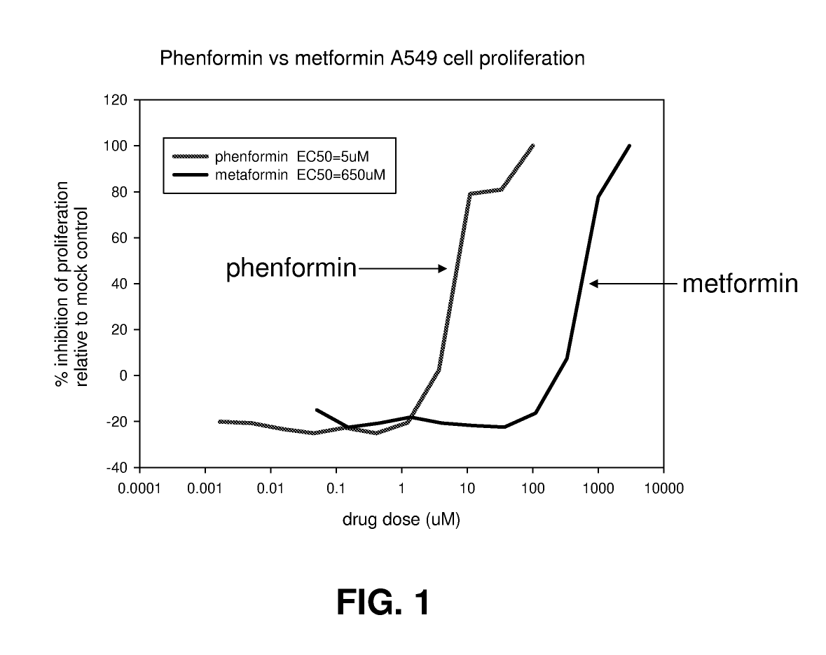

FIG. 1 shows that phenformin effectively inhibited A549 cell proliferation. Phenformin (EC50=5 μM) was about 112-fold more sensitive than metformin (EC50=650 μM) in inhibiting the proliferation of A549 cells.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only. Additionally, some other embodiments are encompassed within the claims.

1-13. (canceled)

14. A method of treating a patient suffering from a cancer characterized by cells having an aberrant genotype for EGFR or KRAS, comprising administering to the patient in need thereof a therapeutically effective amount of an AMPK activator.

15. The method of claim 14, wherein the AMPK activator is a direct activator.

16. (canceled)

17. The method of claim 16, wherein the AMPK activator is phenformin.

18. The method of claim 14, wherein the AMPK activator is administered in conjunction with another anti-cancer agent.

19. The method of claim 14, further comprising the additional step of evaluating the cancer for an aberrant EGFR or KRAS genotype prior to the administration of the AMPK activator.

20. The method of claim 19, wherein the evaluating step comprises DNA sequencing or immunohistochemical analysis.

21. The method of claim 14, wherein the cancer is further characterized by unwanted glycolysis.

22. The method of claim 14, wherein the cancer is further characterized by unwanted glutamine uptake.

23. The method of claim 14, wherein the method further comprises subjecting the patient in need thereof to a second anti-cancer therapeutic treatment selected from the group consisting of radiation therapy, chemotherapy, hormonal therapy or treatment with a targeted cancer compound.

24. The method of claim 23, said second treatment is selected from the group consisting of treatment with taxotere, mitoxantrone, anti-androgen therapy, radiation, anti-estrogen therapy, xeloda, 5-FU, adriamycin, cytoxan, taxol, taxotere, avastin, herceptin, temodar and tarceva.

25. The method of claim 14, wherein the cancer is cancer of the colon, lung, pancreas, prostate, breast or brain.

26. The method of claim 14, wherein the patient is suffering from at least one tumor caused by the cancer.

27. A method of promoting apoptosis of a cell characterized as having an aberrant EGFR or KRAS genotype, comprising contacting the cell with an AMPK activator.

28. The method of claim 27, wherein the AMPK activator is a direct activator.

29. (canceled)

30. The method of claim 29, wherein the AMPK activator is phenformin.

31. The method of claim 27, wherein the cell is a cancer cell.

32. The method of claim 31, wherein the cancer is cancer of the colon, lung, pancreas, prostate, breast or brain.

33. The method of claim 32, wherein the AMPK activator is used in conjunction with another anti-cancer agent.

34. The method of claim 33, wherein the method further comprises subjecting the characterized cell to a second anti-cancer therapeutic treatment selected from the group consisting of radiation therapy, chemotherapy, hormonal therapy or treatment with a targeted cancer compound.

35. The method of claim 34, wherein said second treatment is selected from the group consisting of treatment with taxotere, mitoxantrone, anti-androgen therapy, radiation, anti-estrogen therapy, xeloda, 5-FU, adriamycin, cytoxan, taxol, taxotere, avastin, herceptin, temodar and tarceva.

23. The method of claim 14, wherein the method further comprises subjecting the patient in need thereof to a second anti-cancer therapeutic treatment selected from the group consisting of radiation therapy, chemotherapy, hormonal therapy or treatment with a targeted cancer compound.

24. The method of claim 23, said second treatment is selected from the group consisting of treatment with taxotere, mitoxantrone, anti-androgen therapy, radiation, anti-estrogen therapy, xeloda, 5-FU, adriamycin, cytoxan, taxol, taxotere, avastin, herceptin, temodar and tarceva.

25. The method of claim 14, wherein the cancer is cancer of the colon, lung, pancreas, prostate, breast or brain.

26. The method of claim 14, wherein the patient is suffering from at least one tumor caused by the cancer.

27. A method of promoting apoptosis of a cell characterized as having an aberrant EGFR or KRAS genotype, comprising contacting the cell with an AMPK activator.

28. The method of claim 27, wherein the AMPK activator is a direct activator.

29. The method of claim 27, wherein the AMPK activator is an indirect activator.

30. The method of claim 29, wherein the AMPK activator is phenformin

31. The method of claim 27, wherein the cell is a cancer cell.

32. The method of claim 31, wherein the cancer is cancer of the colon, lung, pancreas, prostate, breast or brain.

33. The method of claim 32, wherein the AMPK activator is used in conjunction with another anti-cancer agent.
